(19) United States
(12) Patent Application Publication
(10) Pub. No.: US 2026/0306233 A1
(43) Pub. Date: Oct. 8, 2026

(54) ORAL POUCH PRODUCT

(71) Applicants: Elizabeth Ray, Paso Robles, CA (US); Lawrence Ray, Reno, NV (US)

(72) Inventors: Elizabeth Ray, Paso Robles, CA (US); Lawrence Ray, Reno, NV (US)

(21) Appl. No.: 19/639,154

(22) Filed: Apr. 4, 2026

Related U.S. Application Data
(60) Provisional application No. 63/782,780, filed on Apr. 3, 2025.

(51) Int. Cl. A61C 19/06; A61M 31/00

(52) U.S. Cl. CPC A61C 19/063; A61M 31/002

(57) ABSTRACT

An oral pouch product for controlled and prolonged release of fluid within an oral cavity. The product includes a first pouch unit and a second pouch unit, each having a deformable reservoir configured to contain a fluid, and a bridge extending between the pouch units to maintain positioning within the mouth. The pouch units are configured to be positioned between the cheeks and teeth, wherein natural muscular forces of the cheeks apply pressure to the reservoirs to facilitate passive release of the fluid. Each pouch unit may include a refill port and an outlet port for controlled dispensing. The bridge may be adjustable to accommodate different oral anatomies.

### CROSS REFERENCE TO RELATED APPLICATIONS

This application claims priority from a U.S. Provisional Patent Appl. No. 63/782,780, filed on Apr. 3, 2025, which is incorporated herein by reference in its entirety.

### FIELD OF INVENTION

The present invention relates generally to oral delivery devices, and more particularly to an intraoral pouch configured for controlled and prolonged release of a fluid within an oral cavity.

### BACKGROUND

Xerostomia is a medical condition characterized by dryness of the oral cavity resulting from reduced or impaired saliva production. The condition may cause symptoms such as a dry or sticky sensation in the mouth, difficulty in swallowing, altered taste, and a burning sensation. Saliva plays a critical role in maintaining oral health by neutralizing acids, facilitating digestion, and cleansing the oral cavity. A reduction in saliva production increases the risk of dental caries, periodontal disease, and oral infections.

Existing approaches for managing xerostomia are primarily directed toward temporary symptom relief. Such approaches include frequent intake of water, use of artificial saliva substitutes, lozenges, oral sprays, and chewing sugar-free gum to stimulate salivary flow. While these methods may provide short-term relief, they require active and repeated user intervention at regular intervals throughout the day.

These limitations are particularly pronounced during periods of inactivity or sleep, when users are unable to consciously maintain hydration or apply symptomatic treatments. As a result, individuals suffering from xerostomia often experience exacerbated symptoms upon waking, including severe dryness and discomfort.

Accordingly, there exists a need for an improved oral delivery system that can provide controlled and prolonged release of a fluid, such as a saliva substitute, within the oral cavity over an extended duration without requiring continuous user intervention.

### SUMMARY OF THE INVENTION

The following presents a simplified summary of one or more embodiments of the present invention to provide a basic understanding of certain features and advantages thereof. This summary is not an exhaustive overview of all contemplated embodiments and is not intended to identify key or critical elements of the invention or to delineate the scope of the claims. Rather, it introduces selected concepts that are further described in the detailed description.

A principal object of the present invention is to provide an oral pouch product configured to release a fluid, such as a saliva substitute, within an oral cavity over a prolonged duration without requiring active user intervention.

Another object of the present invention is to provide an oral pouch that is simple and convenient to use.

Another object of the present invention is to provide an oral pouch that is economical and suitable for scalable manufacturing.

Another object of the present invention is to provide an oral pouch capable of releasing the fluid over an extended period, including during sleep, to maintain moisture within the oral cavity.

Another object of the present invention is to provide an oral pouch product that is reusable.

The following presents a simplified summary of one or more embodiments of the present invention to provide a basic understanding of certain aspects of the invention. This summary is not intended to identify key or essential elements of the invention or to delineate the scope of the claims.

In one aspect, disclosed is an oral pouch product that operates as a passive, self-actuated delivery system, wherein natural muscular forces within the oral cavity provide a continuous compressive input to the pouch units. In combination with the bridge-based stabilization and flow-regulating outlet structures, the system enables controlled micro-dosing of fluid over an extended duration without requiring active user participation.

In one aspect, disclosed is an oral pouch product configured for placement within an oral cavity of a user for controlled and prolonged release of a fluid. The oral pouch product may include a first pouch unit and a second pouch unit, each comprising a deformable reservoir configured to contain a fluid, and a bridge extending between the pouch units to maintain relative positioning within the oral cavity.

In another aspect, the pouch units may be positioned between the cheeks and adjacent teeth or gums, wherein natural muscular forces of the cheeks apply compressive pressure to the deformable reservoirs, thereby causing passive release of the fluid without requiring active user intervention.

In another aspect, each pouch unit may include a refill port configured to receive a fluid, such as a synthetic saliva or saliva substitute, and an outlet port configured to dispense the fluid at a controlled rate. The refill port may include a one-way valve structure configured to permit ingress of the fluid while substantially preventing leakage.

In another aspect, the bridge may be adjustable and may include engagement features configured to releasably couple with corresponding ports on the pouch units, thereby allowing adjustment of spacing between the pouch units to accommodate different oral anatomies.

In another aspect, the pouch units may be formed from biocompatible elastomeric materials suitable for intraoral use, and may be configured to be refillable and reusable.

In another aspect, the disclosed oral pouch product enables sustained and unattended delivery of the fluid over an extended duration, including during sleep, thereby maintaining moisture within the oral cavity.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 illustrates a perspective view of the oral pouch product, according to an exemplary embodiment of the present invention.

FIG. 2 illustrates the right pouch unit, according to an exemplary embodiment of the present invention.

FIG. 3 illustrates the left pouch unit, according to an exemplary embodiment of the present invention.

FIG. 4 shows an exploded perspective view of a pouch unit, showing a top shell and a bottom shell, according to an exemplary embodiment of the present invention.

FIG. 5 shows a front view of the top and bottom shells of the pouch unit, depicting the external contour and compact profile of the pouch unit, according to an exemplary embodiment of the present invention.

FIGS. 6A-6D illustrate various views of the bridge, including a side view (FIG. 6A), a top view (FIG. 6B), and perspective views (FIGS. 6C and 6D).

### DETAILED DESCRIPTION

The subject matter of the present invention will now be described more fully with reference to the accompanying drawings, which form a part of this disclosure and illustrate specific exemplary embodiments. However, it should be understood that the subject matter may be embodied in various forms and is not limited to the specific embodiments set forth herein. Rather, these embodiments are provided by way of example to convey the scope of the invention. It is intended that the claims encompass a broad range of subject matter, including methods, devices, components, and systems. Accordingly, the following detailed description is not intended to be taken in a limiting sense.

As used herein, the term “exemplary” is intended to mean “serving as an example, instance, or illustration.” Any embodiment described as “exemplary” should not be construed as preferred or more advantageous over other embodiments. Similarly, the expression “embodiments of the present invention” does not imply that all embodiments must include all features, advantages, or modes of operation described.

The terminology used herein is for the purpose of describing particular embodiments only and is not intended to be limiting. As used herein, the singular forms “a,” “an,” and “the” include plural references unless the context clearly dictates otherwise. Furthermore, the terms “comprises,” “comprising,” “includes,” and/or “including” specify the presence of stated features, integers, steps, operations, elements, or components, but do not preclude the presence or addition of one or more other features, integers, steps, operations, elements, components, and/or groups thereof.

The following detailed description sets forth the best currently contemplated modes for carrying out exemplary embodiments of the invention. This description is not intended to be limiting, but rather to illustrate the general principles of the invention. The claims of any issued patent will define the scope of the invention.

The present invention relates to an intraoral pouch product configured to be positioned within an oral cavity of a user for controlled and prolonged release of a fluid. In certain embodiments, the fluid may comprise a saliva substitute for alleviating symptoms associated with xerostomia. The oral pouch product is particularly suitable for use during periods of inactivity, such as sleep, wherein the device provides sustained moisture within the oral cavity and reduces discomfort associated with extended dryness.

The oral pouch product may be positioned between a cheek and teeth or gums of the user. When positioned, natural muscular forces of the cheek exert a compressive pressure on the pouch, thereby facilitating controlled release of the fluid from the pouch into the oral cavity. The pouch may include a reservoir configured to store the fluid and a dispensing feature configured to regulate release. In certain embodiments, the oral pouch product is configured to be refillable and reusable, thereby enhancing cost-effectiveness and user convenience.

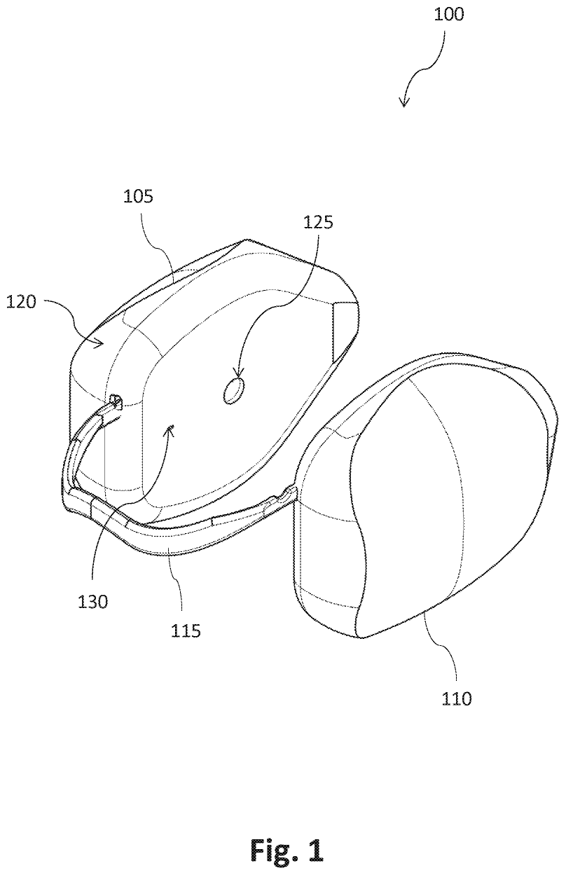

Referring to FIG. 1, a perspective view of an oral pouch product 100 is illustrated. The oral pouch product 100 includes a first pouch unit 105, a second pouch unit 110, and a bridge 115 extending between the first pouch unit 105 and the second pouch unit 110. The bridge 115 is configured to mechanically couple the pouch units and maintain their relative positioning within an oral cavity of a user.

In certain embodiments, the bridge 115 may be adjustable in length and/or flexibility to accommodate different oral anatomies. During use, the first pouch unit 105 may be positioned between a first cheek and adjacent teeth or gums, while the second pouch unit 110 may be positioned between an opposing cheek and corresponding teeth or gums. The bridge 115 assists in retaining the pouch units in position without substantially interfering with normal oral functions such as speaking or breathing.

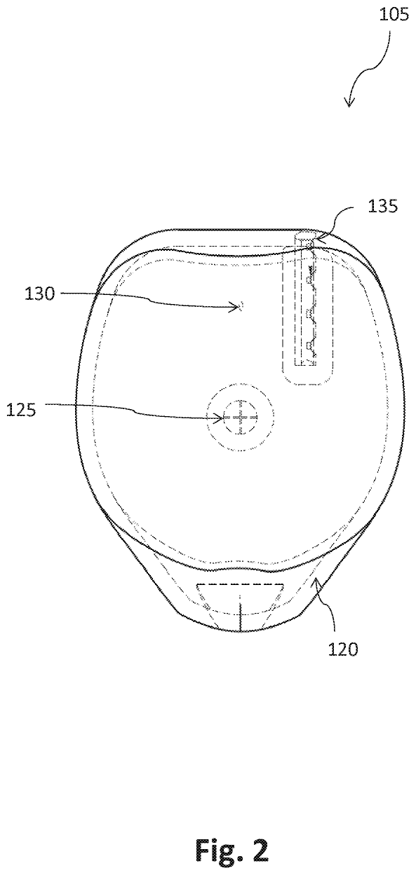

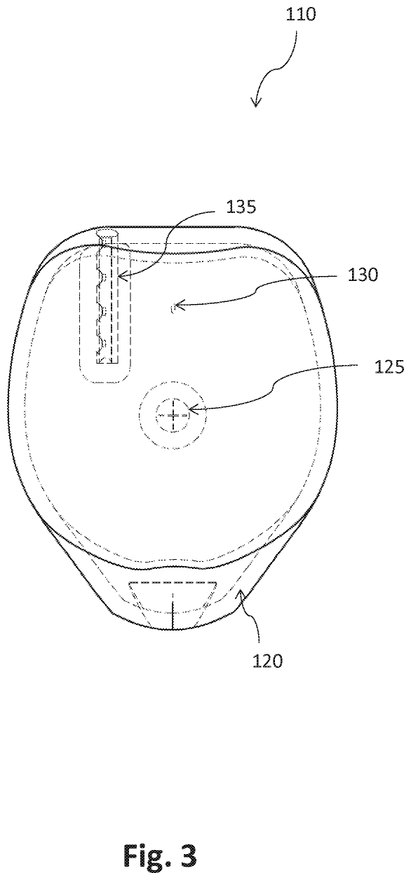

Referring to FIGS. 2 and 3, the first pouch unit 105 and the second pouch unit 110 are illustrated, respectively. Each pouch unit includes a reservoir 120 configured to contain a fluid. The reservoir 120 may be formed from a deformable or elastomeric material such that application of external pressure, including pressure exerted by surrounding oral musculature, causes controlled release of the fluid from the reservoir.

In certain embodiments, the reservoir 120 may be fabricated from a biocompatible, medical-grade material suitable for intraoral use. An outer surface of the reservoir may include a coating configured to be substantially neutral in taste and odor to enhance user comfort. An inner surface of the reservoir may be smooth and optionally treated or impregnated with antimicrobial agents to inhibit microbial growth and maintain hygiene during repeated use.

As shown in the drawings, each pouch unit may have a generally hemi-spheroidal or partially spheroidal shape. The geometry of the pouch unit is configured to be comfortably and securely positioned between a cheek and adjacent teeth or gums of a user. An outer wall of each pouch unit may be contoured to conform to the inner surface of the cheek, thereby distributing pressure evenly across the pouch unit. A forward-facing surface of the pouch unit may be relatively planar or flattened to rest against the teeth or gums, thereby improving positional stability during use. The contoured configuration enables uniform compression of the pouch unit by the cheek, facilitating controlled release of fluid from the pouch unit.

Each pouch unit may include a refill port 125 configured to allow introduction of fluid into the reservoir. The refill port 125 may be configured to receive a dispensing tip, syringe, or other filling device for replenishing the fluid within the pouch unit. In certain embodiments, the refill port 125 may include a self-sealing or one-way valve mechanism configured to permit ingress of fluid while substantially preventing unintended leakage or backflow. The refillable configuration allows repeated use of the pouch unit, thereby improving convenience and cost efficiency.

Each pouch unit may further include an outlet port 130 configured to dispense the fluid into the oral cavity. The outlet port 130 may be dimensioned to provide a controlled rate of fluid release. In certain embodiments, the flow rate may be determined based on the size, geometry, or number of outlet openings. In other embodiments, the pouch unit may include alternative flow-regulating structures, such as micro-apertures, porous membranes, slit valves, or capillary channels, to achieve a desired release profile. The rate of discharge may be further influenced by externally applied pressure, including pressure exerted by the cheek during use.

The oral pouch product is configured such that natural muscular forces of the cheek apply compressive pressure to a deformable reservoir, thereby causing controlled expulsion of fluid without active user intervention. The release mechanism is passive and responsive to physiological forces within the oral cavity, eliminating the need for pumps, electronics, or user-triggered actuation.

Each pouch unit is configured to provide gradual and sustained release of fluid over an extended duration, including during sleep. The release rate may be governed by one or more outlet ports or flow-regulating structures, enabling micro-dosing of fluid to maintain oral moisture over time.

The system includes two pouch units positioned on opposing sides of the oral cavity, interconnected by a bridge structure that maintains spatial alignment and prevents displacement during use. The bridge may be flexible and/or adjustable to accommodate different oral anatomies while ensuring retention without adhesive or fixation mechanisms.

Each pouch unit 105, 110 may include a bridge port 135 disposed on a rear-facing portion of the pouch unit. The bridge port 135 is configured to receive a corresponding rail 155 of the bridge 115 to mechanically couple the bridge 115 to each pouch unit. The bridge port 135 may have an elongated channel or slot configuration adapted to receive and guide insertion of the rail 155. In certain embodiments, the engagement between the bridge port 135 and the rail 155 may provide a releasable or semi-permanent connection.

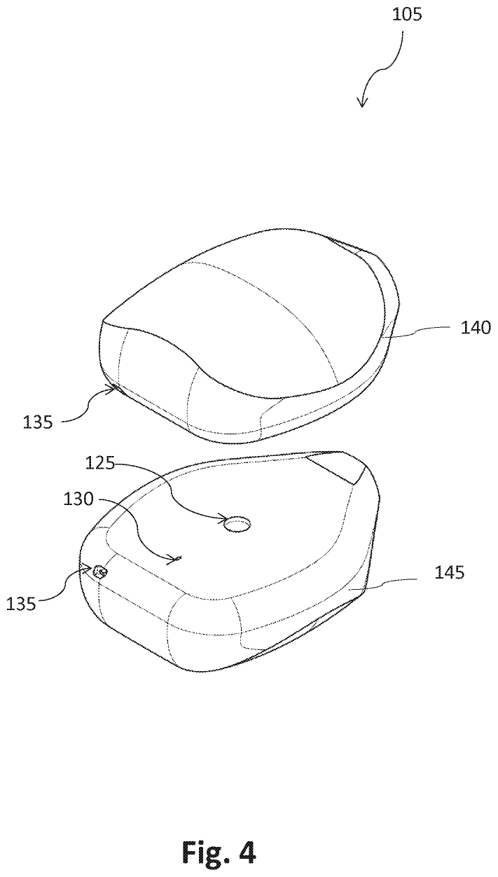

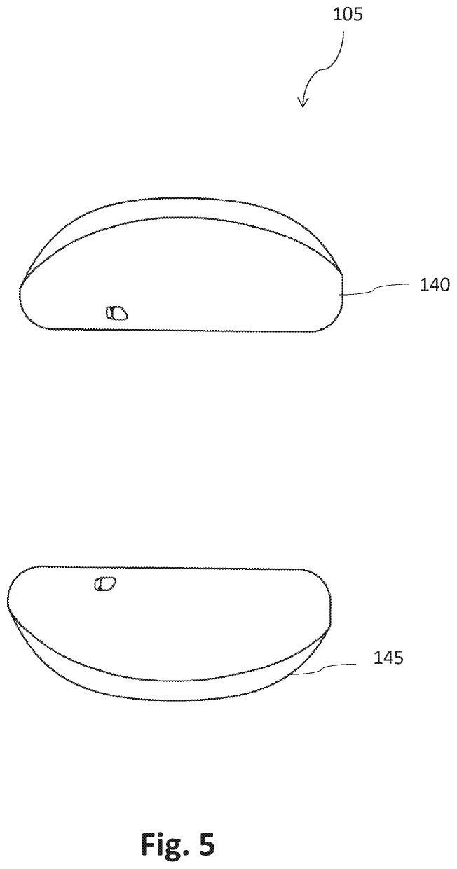

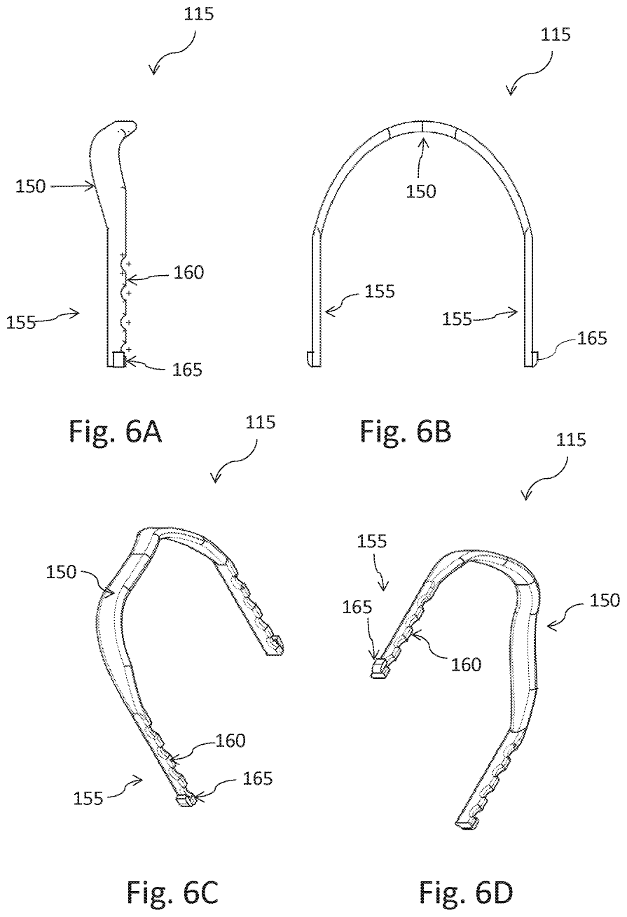

Referring to FIGS. 4 and 5, each pouch unit 105, 110 may be formed from a top shell 140 and a bottom shell 145 that together to define the reservoir 120. Referring to FIGS. 6A-6D, the bridge 115 is illustrated in various views. The bridge 115 may include a generally U-shaped body 150 configured to extend across a portion of the oral cavity. The bridge 115 further includes rail 155 extending from each end of the U-shaped body 150. The rail 155 is configured to be inserted into the bridge port 135 of the respective pouch units 105, 110.

In certain embodiments, the rail 155 may have an elongated profile and may include one or more engagement features, such as spaced-apart ridges 160, configured to provide incremental positional adjustment of the bridge 115 relative to the pouch units. The ridges 160 may engage corresponding internal features within the bridge port 135 to selectively retain the rail 155 at different insertion depths, thereby allowing adjustment of spacing between the pouch units.

The rail 155 may further include a retention feature, such as a knuckle 165 or enlarged portion, configured to resist unintended disengagement of the bridge 115 from the pouch units once inserted. The combination of the ridges 160 and the retention feature 165 provides both adjustability and secure coupling of the bridge 115 to the pouch units.

In certain embodiments, the pouch units are configured to contain a fluid comprising a saliva substitute or synthetic saliva formulation. The fluid may be introduced into each pouch unit using a filling device, such as a transfer syringe including a plastic-tipped or hooked dispensing end. The syringe may be inserted into the refill port 125 to deliver a selected volume of the fluid into the reservoir 120, depending on a desired fill level determined by the user.

filling, the pouch units 105, 110 may be positioned within the oral cavity between the cheeks and adjacent teeth or gums, and connected via the bridge 115. In use, passive elastic forces generated by displacement of the cheek tissues apply a substantially continuous compressive force on the pouch units. This compressive force increases internal pressure within the reservoirs 120, thereby causing the fluid to be dispensed through the outlet port 130 in a controlled manner over time.

In certain embodiments, the refill port 125 may be configured as a self-sealing inlet having a one-way valve functionality. The geometry of the refill port 125 may permit ingress of fluid during filling while substantially preventing egress during use. The one-way valve behavior may be achieved through a slit configuration, elastomeric deformation, or other valve-like structures analogous in function to biological valves.

The pouch units may be formed from biocompatible materials suitable for intraoral use and compliant with applicable standards, including ISO standards for medical or oral-contact materials. In certain embodiments, the material may comprise an elastomer having an elongation or displacement capability of at least approximately 20 percent, thereby allowing deformation under cheek pressure and expansion during filling. The material may further be configured to provide a smooth external finish to enhance user comfort and reduce irritation.

Exemplary materials suitable for forming the pouch units include silicone-based materials, such as liquid silicone rubber (LSR) and medical-grade silicone. However, other biocompatible elastomers having similar mechanical and surface properties may also be used.

In certain embodiments, each pouch unit may have approximate dimensions of about 42 mm in length, 34 mm in height, and 14 mm in width in an unexpanded state. Due to the elastic properties of the material, the pouch unit may expand to dimensions of approximately 50 mm in length, 41 mm in height, and 17 mm in width when filled. It should be understood that the dimensions may vary depending on material properties, fill volume, and user-specific anatomical considerations.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An oral pouch product configured for placement within an oral cavity of a user, the oral pouch product comprising:
a first pouch unit comprising a first deformable reservoir configured to contain a fluid;
a second pouch unit comprising a second deformable reservoir configured to contain the fluid; and
a bridge extending between the first pouch unit and the second pouch unit and configured to maintain relative positioning of the first pouch unit and the second pouch unit within the oral cavity,
wherein each of the first pouch unit and the second pouch unit is configured to be positioned between a cheek and adjacent teeth or gums of the user, and
wherein the first deformable reservoir and the second deformable reservoir are configured to release the fluid in response to compressive forces applied by oral musculature of the user.

2. The oral pouch product of claim 1, wherein each pouch unit further comprises an outlet port configured to provide controlled release of the fluid.

3. The oral pouch product of claim 1, wherein each pouch unit further comprises a refill port configured to receive the fluid.

4. The oral pouch product of claim 1, wherein each pouch unit has a generally hemi-spheroidal shape.

5. The oral pouch product of claim 1, wherein each pouch unit includes a contoured outer surface configured to conform to an inner surface of the cheek.

6. The oral pouch product of claim 1, wherein each pouch unit includes a generally planar surface configured to engage the teeth or gums.

7. The oral pouch product of claim 1, wherein the bridge is adjustable in length.

8. The oral pouch product of claim 1, wherein each pouch unit comprises a bridge port configured to receive a rail of the bridge.

9. The oral pouch product of claim 8, wherein the rail comprises a plurality of spaced-apart ridges configured to provide incremental positional adjustment.

10. The oral pouch product of claim 1, wherein the fluid comprises a saliva substitute.

11. A method of delivering a fluid within an oral cavity of a user, the method comprising:
positioning a first pouch unit between a first cheek and adjacent teeth or gums;
positioning a second pouch unit between a second cheek and adjacent teeth or gums;
maintaining the first pouch unit and the second pouch unit in position using a bridge extending therebetween; and
releasing the fluid from at least one of the first pouch unit or the second pouch unit in response to compressive forces applied by oral musculature.
